SEQUENCE LISTING (10 sequences)

SEQ ID NO: 1 - RNA, 49 nt, Human immunodeficiency virus type 1
acgcaggacucggcuugcugaagcgcgcacggcaagaggcgaggggcgg

SEQ ID NO: 2 - DNA, 20 bp, Human immunodeficiency virus type 1
  misc_feature (1)..(20): All internucleotide bonds are phosphorothioate bonds.
cttcagcaagccgagtcctg

SEQ ID NO: 3 - DNA, 20 bp, Human immunodeficiency virus type 1
  misc_feature (1)..(20): All internucleotide bonds are phosphorothioate bonds.
cgcttcagcaagccgagtcc

SEQ ID NO: 4 - DNA, 20 bp, Human immunodeficiency virus type 1
  misc_feature (1)..(20): All internucleotide bonds are phosphorothioate bonds.
tgccgtgcgcgcttcagcaa

SEQ ID NO: 5 - DNA, 20 bp, Human immunodeficiency virus type 1
  misc_feature (1)..(20): All internucleotide bonds are phosphorothioate bonds.
cctcgcctcttgccgtgcgc

SEQ ID NO: 6 - DNA, 19 bp, Human immunodeficiency virus type 1
  misc_feature (1)..(19): All internucleotide bonds are phosphorothioate bonds.
ccctcgcctcttgccgtgc

SEQ ID NO: 7 - DNA, 20 bp, Artificial Sequence
  Description of Artificial Sequence an artificially synthesized random sequence
  misc_feature (1)..(20): All internucleotide bonds are phosphorothioate bonds.
gcgtcatacgtcttgcggac

SEQ ID NO: 8 - DNA, 20 bp, Artificial Sequence
  Description of Artificial Sequence an artificially synthesized random sequence
  misc_feature (1)..(20): All internucleotide bonds are phosphorothioate bonds.
cggttacgccatgcgcatcg

SEQ ID NO: 9 - DNA, 28 bp, Human immunodeficiency virus type 1
  misc_feature (1)..(28): All internucleotide bonds are phosphorothioate bonds.
cgctctcgcacccatctctctccttcta

SEQ ID NO: 10 - DNA, 37 bp, Human immunodeficiency virus type 1
  misc_feature (1)..(37): All internucleotide bonds are phosphorothioate bonds.
ttttcttttggggggtttttttccccccttttctttt

(19) United States
(12) Patent Application Publication
(10) Pub. No.: US 2007/0082862 A1
(43) Pub. Date: Apr. 12, 2007

(54) Novel antisense oligonucleotide and anti-hiv agent

(76) Inventors: Hiroshi Takaku, Chiba (JP); Naoko Kurosaki, Tokyo (JP)

Correspondence Address: HESLIN ROTHENBERG FARLEY & MESITI PC, 5 COLUMBIA CIRCLE, ALBANY, NY, 12203, US

(21) Appl. No.: 10/549,601

(22) PCT Filed: Mar. 19, 2004
(86) PCT No.: PCT/JP04/03776

§ 371 Date: Aug. 8, 2006

(30) Foreign Application Priority Data
Mar. 19, 2003 (JP) 2003-076254

(51) Int. Cl. A61K 48/00; C07H 21/02

(52) U.S. Cl. 514/44; 514/81; 536/23.1

(57) ABSTRACT

A novel antisense oligonucleotide that targets at DIS (Dimerization Initiation Site) region of HIV-1; and an anti-HIV agent (HIV curative and/or preventive agent) comprising the same. This anti-HIV agent realizes effective treatment and/or prevention for HIV infection.

### TECHNICAL FIELD

The present invention relates to a novel antisense oligonucleotide and an anti-HIV agent (an agent for treating and/or preventing HIV).

### BACKGROUND ART

As an antiviral agent for a human immunodeficiency virus (hereinafter referred to as HIV), an approach on the basis of an antisense method in which a gene is targeted is known. The antisense method is a technique in which an oligonucleotide having a nucleotide sequence complementary to a target gene is used to inhibit a transcription of the target gene, a splicing of an mRNA, and/or a translation to a protein, thereby specifically inhibiting an expression of the viral protein. One of the most important objects in the antisense method is a selection of a site to be targeted.

As antisense oligonucleotides useful as an active ingredient of an anti-HIV agent, for example, oligonucleotides comprising a nucleotide sequence complementary to each nucleotide sequence of a CXCR4 gene or a CCR5 gene [Japanese Unexamined Patent Publication (Kokai) No. 11-292795 (Patent reference 1)], or antisense RNAs for an env portion, env and pol portions, or env, pol, and gag portions [Japanese Translation Publication (Kohyo) No. 2001-502884 (Patent reference 2)] are known.
Patent reference 1: Japanese Unexamined Patent Publication (Kokai) No. 11-292795
Patent reference 2: Japanese Translation Publication (Kohyo) No. 2001-502884

### DISCLOSURE OF INVENTION

An object of the present invention is to provide a novel oligonucleotide useful as an active ingredient of an anti-HIV agent capable of effectively treating and/or preventing an HIV infection, and an anti-HIV agent (an agent for treating and/or preventing HIV) containing the same.

The object may be attained by the present invention, that is, by an oligonucleotide consisting of a nucleotide sequence complementary to a nucleotide sequence consisting of at least 15 successive nucleotides in the nucleotide sequence consisting of nucleotides 6-44 of SEQ ID NO: 1.

The present invention relates to an oligonucleotide comprising a nucleotide sequence which specifically hybridizes to a nucleotide sequence consisting of at least 15 successive nucleotides in the nucleotidec sequence consisting of nucleotides 6-44 of SEQ ID NO: 1.

The present invention relates to a pharmaceutical composition comprising the oligonucleotide and a pharmaceutically or veterinarily acceptable carrier or diluent.

The present invention relates to an anti-HIV agent comprising as an active ingredient the oligonucleotide.

The present invention relates to a pharmaceutical composition for treating or preventing HIV, comprising the oligonucleotide and a pharmaceutically or veterinarily acceptable carrier or diluent.

The present invention relates to a method for treating or preventing HIV, comprising administering to a subject in need thereof the oligonucleotide in an amount effective therefor.

The present invention relates to the use of the oligonucleotide in the manufacture of an anti-HIV agent or a pharmaceutical composition for treating or preventing HIV.

The term “HIV” as used herein means a human immunodeficiency virus, including HIV-1 and variants thereof.

### BRIEF DESCRIPTION OF THE DRAWINGS

FIG. 1 illustrates the relationship between the nucleotide sequences of S-ODNs used in Examples and the DIS region of HIV-1 as the target sequence.

FIG. 2 is a graph showing amounts of an HIV-1 p24 antigen in culture supernatants of 293T cells transfected with various concentrations of S-ODNs (0.1 μmol/L, 0.5 μmol/L, and 1.0 μmol/L) and plasmid pNL4-3 (1 μg).

FIG. 3 is a graph showing amounts of an HIV-1 p24 antigen in intracellular proteins of 293T cells transfected with various concentrations of S-ODNs (0.1 μmol/L, 0.5 μmol/L, and 1.0 μmol/L) and plasmid pNL4-3 (1 μg).

FIG. 4 is a graph showing a luciferase activity in intracellular proteins of 293T cells transfected with various S-ODNs (1.0 μmol/L) and plasmid pNL-luc (1 μg).

FIG. 5 is a graph showing an expression level of intracellular HIV-1 mRNA gene, detected by RT-PCR, in 293T cells transfected with various S-ODNs (1.0 μmol/L) and plasmid pNL-luc (1 μg).

FIG. 6 schematically illustrates the HIV-1 genomic RNA and the structure of the 5′ terminus thereof.

FIG. 7 schematically illustrates (A) the structure of the 5′ terminus of the HIV-1 genomic RNA and (B) the secondary RNA structure model thereof.

FIG. 8 schematically illustrates the translational mechanisms of (A) HIV-1 genomic RNA and (B) viral mRNA.

FIG. 9 is a graph showing an antiviral activity with respect to human peripheral blood lymphocytes as the target.

FIG. 10 is a graph showing an antiviral activity with respect to human peripheral blood lymphocytes as the target.

### BEST MODE FOR CARRYING OUT THE INVENTION

Hereinafter the present invention will be explained.

[1] Oligonucleotide of the Present Invention

The oligonucleotide of the present invention includes
(1) an oligonucleotide consisting of a nucleotide sequence complementary to a nucleotide sequence consisting of at least 15 successive nucleotides in the nucleotide sequence consisting of nucleotides 6-44 of SEQ ID NO: 1, and
(2) an oligonucleotide comprising a nucleotide sequence which specifically hybridizes to a nucleotide sequence consisting of at least 15 successive nucleotides in the nucleotide sequence consisting of nucleotides 6-44 of SEQ ID NO: 1.

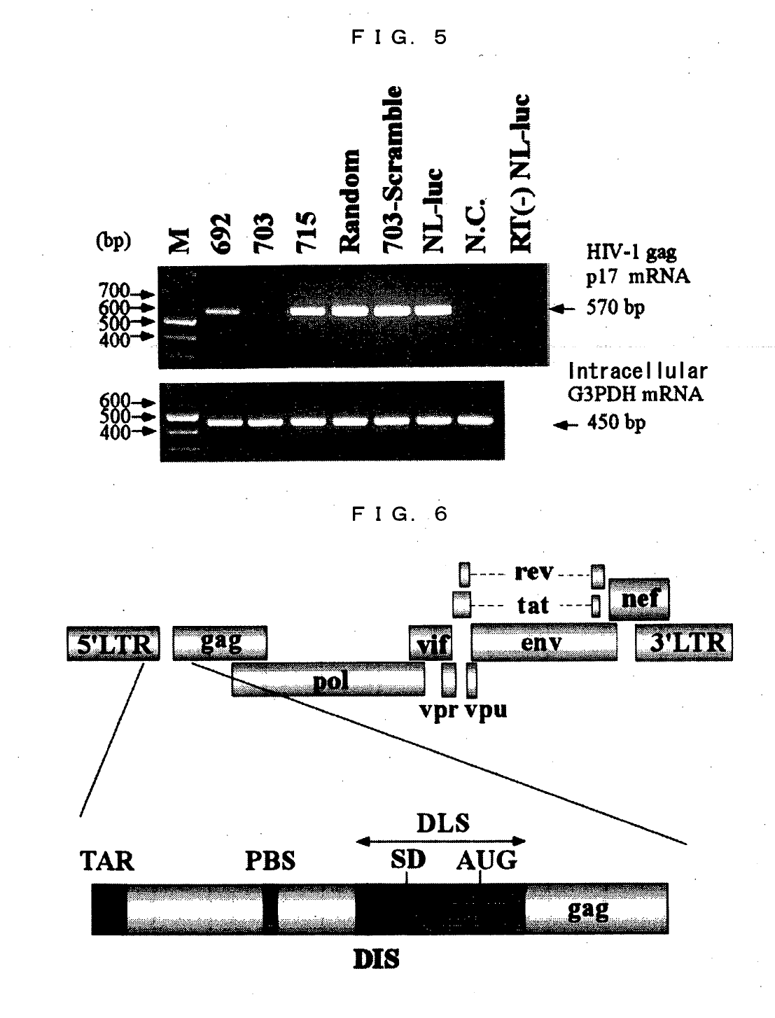

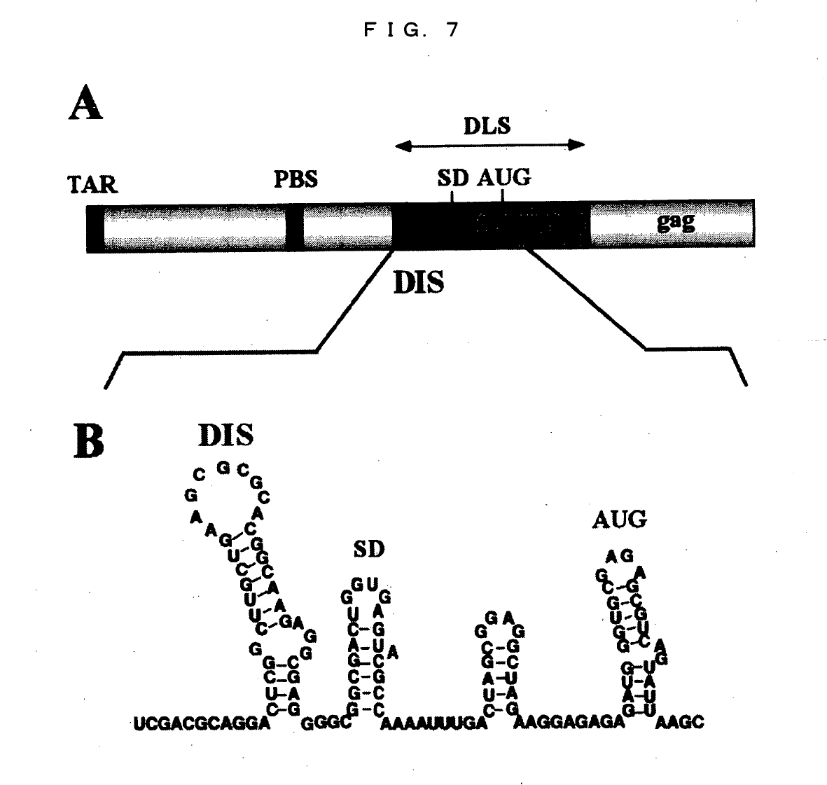

The nucleotide sequence of SEQ ID NO: 1 is a nucleotide sequence of a DIS (Dimerization Initiation Site) region and an adjacent region thereof. The DIS region is located in a DLS (Dimer Linkage Structure) region of HIV-1, and corresponds to the nucleotide sequence consisting of nucleotides 9-43 of SEQ ID NO: 1 (see FIGS. 6 and 7).

As shown in FIGS. 6 and 7, the DLS of HIV-1 is located, downstream of 5′ LTR, at a region containing the initiation codon of a gag gene. It was reported, at the beginning of 1995, that the untranslated flanking sequence was important for a replication step of HIV-1 (Muriaux, D. et al., J. Biol. Chem., 270, 8209-16, 1995), and that the DIS region located in the DLS region was an essential site for a replication of HIV-1, on the basis of in vitro experiments (Berkhout, B. and van Wamel, J. L., RNA, 6, 282-95, 2000; and Damgaard, C. K. et al., Nucleic Scids Res., 26, 3667-76, 1998). The DIS region is located upstream of the initiation codon and SD (Splicing Donor Site) of the gag gene, and it is considered that a stem-loop structure is formed.

However, an antisense method in which the DIS region is targeted was not reported, and it was also not reported whether or not such an approach was effective. In antisense methods, it is well-known that, if a target sequence is essential for a replication or growth of a virus, an antisense oligonucleotide for the target sequence is not necessarily effective as an antiviral agent. Therefore, it could not be predicted, even by those skilled in the art, whether or not antisense oligonucleotides for the DIS region, which was considered to be important for an HIV production, are effective.

As shown in Examples described below, the present inventors designed five phosphorothioate oligonucleotides (S-ODNs; Anti-692, Anti-694, Anti-703, Anti-713, and Anti-715), as antisense oligonucleotides for the DIS region and an adjacent region thereof (the nucleotide sequence of SEQ ID NO: 1), and examined an activity of inhibiting the HIV-1 production. As a result., it was found that only three antisense oligonucleotides (Anti-694, Anti-703, and Anti-713) exhibited an excellent activity of inhibiting the HIV-1 production, dose-dependent.

In this connection, as other target sites considered to be important for the HIV production, an antisense oligonucleotide (28AS) which targeted at the gag region, and an antisense oligonucleotide (PPT-AS) which targeted at a polypurine tract region important for a reverse transcription of viral genes were examined, and it was confirmed that the antisense oligonucleotides did not exhibit the activity of inhibiting the HIV-1 production.

The result that only the DIS region was effective as a target, among plural target regions considered to be important for an HIV production, was not predicted before the experiments, and was unexpected. Further, although it was reported that the DIS region was important for the HIV production, the result that Anti-692 and Anti-715 (the antisense oligonucleotides which contained the DIS region as a target sequence) did not inhibit the HIV production was unexpected, from the view point of the importance of the DIS region.

In the oligonucleotide of the present invention consisting of a nucleotide sequence complementary to a nucleotide sequence consisting of at least 15 successive nucleotides in the nucleotide sequence consisting of nucleotides 6-44 of SEQ ID NO: 1, the number of nucleotides is not particularly limited, so long as it is 15 nucleotides or more. The nucleotide number that ensures a specific hybridization to a target region is preferably 15 nucleotides or more, more preferably 18 nucleotides or more, most preferably 20 nucleotides or more. The nucleotide number that ensures a membrane penetration is preferably 30 nucleotides or less, more preferably 28 nucleotides or less, most preferably 25 nucleotides or less. The oligonucleotide of the present invention consists of preferably 15 to 30 nucleotides, more preferably 18 to 28 nucleotides, still further preferably 20 to 25 nucleotides, most preferably 20 nucleotides.

The oligonucleotide of the present invention comprising a nucleotide sequence which specifically hybridizes to a nucleotide sequence consisting of at least 15 successive nucleotides in the nucleotide sequence consisting of nucleotides 6-44 of SEQ ID NO: 1 is not particular limited, so long as it can hybridize to the target nucleotide sequence under the same conditions as those in a living body (for example, in a liquid medium at 37° C.) and exhibits an anti-HIV activity. The oligonucleotide of the present invention includes, for example,
an oligonucleotide exhibiting an anti-HIV activity, and comprising a nucleotide sequence complementary to a nucleotide sequence consisting of at least 15 successive nucleotides in the nucleotide sequence consisting of nucleotides 6-44 of SEQ ID NO: 1; and
an oligonucleotide exhibiting an anti-HIV activity, and comprising a nucleotide sequence complementary to a nucleotide sequence in which one or several nucleotides (preferably one or two nucleotides, more preferably one nucleotide) are substituted, inserted, and/or deleted in a nucleotide sequence consisting of at least 15 successive nucleotides in the nucleotide sequence consisting of nucleotides 6-44 of SEQ ID NO: 1.

As the oligonucleotide exhibiting an anti-HIV activity, and comprising a nucleotide sequence complementary to a nucleotide sequence consisting of at least 15 successive nucleotides in the nucleotide sequence consisting of nucleotides 6-44 of SEQ ID NO: 1, there may be mentioned, for example,
an oligonucleotide exhibiting an anti-HIV activity, and consisting of a nucleotide sequence in which one or several nucleotides (preferably 1 to 10 nucleotides, more preferably 1 to 5 nucleotides, still further preferably 1 to 3 nucleotides, still further preferably 1 or 2 nucleotides, most preferably 1 nucleotide) are added to the 5′ terminus and/or the 3′ terminus of a nucleotide sequence complementary to a nucleotide sequence consisting of at least 15 successive nucleotides in the nucleotide sequence consisting of nucleotides 6-44 of SEQ ID NO: 1; more particularly,
an oligonucleotide consisting of any one of the nucleotide sequences of SEQ ID NOS: 3 to 5 wherein each of the internucleotide bonds between nucleosides may be independently a phosphodiester bond or a modified phosphodiester bond; or
an oligonucleotide exhibiting an anti-HIV activity, and consisting of a nucleotide sequence in which one or several nucleotides (preferably 1 to 10 nucleotides, more preferably 1 to 5 nucleotides, still further preferably 1 to 3 nucleotides, still further preferably 1 or 2 nucleotides, most preferably 1 nucleotide) are added to the 5′ terminus and/or the 3′ terminus of any one of the nucleotide sequences of SEQ ID NOS: 3 to 5.

In this connection, as the nucleotide(s) added to the 5′ terminus and/or the 3′ terminus thereof, a nucleotide complementary to a corresponding nucleotide in the nucleotide sequence of SEQ ID NO: 1 is preferable.

The oligonucleotide of the present invention may be prepared from deoxyribonucleosides, ribonucleosides, and/or modified ribonucleosides thereof, such as 2′-O-modified ribonucleosides, so long as the resulting oligonucleotide can function as an antisense oligonucleotide. A preferable modified ribonucleoside is 2′-O-methylribonucleoside, in view of a strong binding property thereof with the base sequence of the target.

Therefore, the oligonucleotide of the present invention may be an oligoribonucleotide (RNA) composed of ribonucleosides and/or modified ribonucleosides, an oligodeoxyribonucleotide (DNA) composed only of deoxyribonucleosides, or a chimera oligoribo/deoxyribonucleotide (RNA/DNA) composed of ribonucleosides (and/or modified ribonucleosides) and deoxyribonucleosides.

In the oligonucleotide of the present invention, internucleotide bonds between nucleosides may be independently a phosphodiester bond or a modified phosphodiester bond. The modified phosphodiester bond may be, for example, a methylphosphonate bond wherein one of two non-crosslinked oxygen atoms in the phosphodiester bond is replaced with a methyl group; a phosphoroamidate bond wherein one of two non-crosslinked oxygen atoms in the phosphodiester bond is replaced with an amino group or a substituted amino group; or a phosphorothioate bond wherein one of two non-crosslinked oxygen atoms in the phosphodiester bond is replaced with a sulfur atom. The oligonucleotide may contain one or more modified phosphodiester bonds as above in one or more internucleotide bonds between nucleosides.

The phosphodiester bond is preferable, from the standpoints of the specificity to a nucleotide sequence, an easy procedure for preparation, and a cost of production, and the modified phosphodiester bond is preferable, from the standpoints of a stability of the double-stranded chain, a resistance to a nuclease, a penetrating property through a cell membrane, a low cytotoxicity, and a moderate metabolizability. Further, the phosphorothioate bond is more preferable from the standpoint of stability in a living body.

The oligonucleotide used in the present invention may be prepared by known methods. For example, the oligonucleotide may be prepared by an automated DNA/RNA synthesizer in accordance with a conventional phosphodiester method or phosphotriester method, such as an H-phosphonate method or a phosphoramidite method, except for a site to which a 2′-O-methylribonucleotide or a phosphorothioate bond is introduced.

The oligonucleotide having phosphorothioate bonds may be prepared, for example, using a 15% N,N,N′,N′-tetraethylthiorumdisulfide/acetonitrile solution instead of a water/iodine/pyridine solution that is an oxidizing agent used in a conventional synthesis of polynucleotide.

The oligonucleotide having 2′-O-methylribonucleotides may be prepared, for example, by an automated DNA/RNA synthesizer in accordance with the phosphoramidite method, using a 5′-dimethoxytrityl-2′-O-methylribonucleoside-3′-[(2-cyanoethyl)-(N,N-diisopropyl)]-phosphoroamidite unit.

[2] Anti-HIV Agent of the Present Invention

The oligonucleotide of the present invention exhibits an anti-HIV activity, and is useful as an active ingredient of the anti-HIV agent according to the present invention. The term “anti-HIV activity” as used herein means, but is by no means limited to, for example, an activity of inhibiting a production of HIV (particularly HIV-1), or an activity of suppressing an expression of mRNAs or proteins of HIV (particularly HIV-1).

The anti-HIV agent of the present invention may contain as the active ingredient the oligonucleotide of the present invention alone, or optionally with a pharmaceutically or veterinarily acceptable known carrier or diluent, and may be prepared in accordance with known formulating techniques (for example, JP 11-292795) of pharmaceutical compositions containing an antisense oligonucleotide.

As the anti-HIV agent of the present invention, there may be mentioned, for example, an anti-HIV agent containing the oligonucleotide of the present invention, an anti-HIV agent containing the oligonucleotide of the present invention and a liposome stable in blood, or an anti-HIV agent containing an vector comprising the oligonucleotide of the present invention.

The anti-HIV agent of the present invention may be administered via any of an oral, parenteral or local route. The dose may vary with the species of the subject to be treated (a mammal, particularly a human), a response of the subject to the medicine, a formulation of the medicine, an administration time, an interval of administrations, or the like, but may generally be about 500 mg to about 5000 mg/day.

The anti-HIV agent of the present invention may be administered in the form of the oligonucleotide of the present invention, the liposome stable in blood, or the vector comprising the oligonucleotide of the present invention, and optionally, with a pharmaceutically acceptable known carrier or dilute via any of the oral, parenteral or local routes, once or a multiple of times. The anti-HIV agent of the present invention may be variously formulated to produce, for example, tablets, capsules, lozenges, troches, hard candies, powders, sprays, creams, ointments, suppositories, jellies, gels, pastes, lotions, salves, aqueous suspensions, solutions for injection, elixirs, syrups, or the like.

### EXAMPLES

The present invention now will be further illustrated by, but is by no means limited to, the following Examples.

### Example 1

### Preparation of Antisense Oligonucleotides

In this example, three antisense oligonucleotides and two random oligonucleotides were synthesized on the basis of the nucleotide sequence of the DIS (Dimerization Initiation Site) located in the DLS (Dimer Linkage Structure) region of HIV-1, as shown in FIG. 1. All oligonucleotides were DNAs in which all internucleotide bonds were phosphorothioate bonds, i.e., phosphorothioate oligonucleotides (S-ODNs).

In FIG. 1, each of the numbers in the nucleotide sequence of SEQ ID NO: 1, such as “690” or “700”, are nucleotide numbers, when the U3 region in the 5′ LTR of a HIV-1 NL432 strain is regarded as the starting point. The terms “Stem”, “Loop”, and “Bulge” mean a stem region, a loop region, and a bulge region, respectively.

An oligonucleotide “Anti-703” (SEQ ID NO: 4) was synthesized as the oligonucleotide of the present invention. Oligonucleotides “Anti-692” (SEQ ID NO: 2) and “Anti-715” (SEQ ID NO: 6) were synthesized as oligonucleotides for comparison. Oligonucleotides “Random” (SEQ ID NO: 7) and “703-Scramble” (SEQ ID NO: 8) were synthesized as control oligonucleotides not having a sequence complementary to the HIV-1 RNA. The oligonucleotide “703-Scramble” shows the same GC content as that of the oligonucleotide “Anti-703” of the present invention.

### Example 2

### Evaluation of S-ODNs for Anti-HIV Activity Using Plasmid pNL4-3

(1) Transfection with S-ODNs and Plasmid pNL4-3

In this example, a 293T cell (ATCC No. CRL-11268) derived from a human kidney was transfected with an HIV-1 expression vector, plasmid pNL4-3 (Adach, a. et al., J. Virol., 59, 248, 1986), to evaluate S-ODNs prepared in Example 1 with respect to an activity of inhibiting an HIV-1 production.

When the 293T cells are transfected with plasmid pNL4-3, an LTR promoter thereof causes an expression of genes, and HIV-1 is produced. In this example, an amount of a HIV-1 protein, a p24 antigen, produced in the cell and secreted to the culture supernatant was measured.

The transfection of the 293T cell with S-ODNs and plasmid pNL4-3 was carried out using a commercially available transfection reagent (FuGENE™6 Transfection Reagent; Boehringer Mannheim, L. L. C, USA) in accordance with a protocol attached thereto.

More particularly, the 293T cells (10⁵ cells/2 mL medium/well) were added to each well of a 6-well plate, and incubated in an incubator (at 37° C. and 5% CO₂) overnight. As the medium, a Dulbecco's modified Eagle's medium (D-MEM) supplemented with 10% fetal bovine serum (FBS) was used.

After the overnight incubation, it was confirmed that the cells adhered to the wells. After the medium was removed from each well, 900 μL of a serum-free RPMI-1640 medium was added, and the plate was placed in the incubator (at 37° C. and 5% CO₂). Further, 100 μL of each FuGENE™6/S-ODN solution, which had been previously prepared by diluting the transfection reagent (FuGENE™6) and each S-ODN with the serum-free RPMI-1640 medium to predetermined concentrations [S-ODN=1 μmol/L, 5 μmol/L, and 10 μmol/L (three levels); FuGENE™6=1/50 dilution], was added to each well. The plate was gently shaken to a mix in each well, and placed in the incubator (at 37° C. and 5% CO₂) for two hours.

After two hours had passed from the addition of the FuGENE™6/S-ODN solution, 110 μL of FuGENE™6/pNL4-3 solution, which had been previously prepared by diluting the transfection reagent (FuGENE™6) and plasmid pNL4-3 with the serum-free RPMI-1640 medium to predetermined concentrations [plasmid pNL4-3=0.01 μg/μL; FuGENE ™6=1/50 dilution], was further added to each well. The plate was gently shaken to a mix in each well, and placed in the incubator (at 37° C. and 5% CO₂) for two hours.

After two hours had passed from the addition of the FuGENE™6/pNL4-3 solution, the medium was removed from each well, and each well was washed with 1 mL of phosphate buffered saline [PBS(−)] three times. After 3 mL of an RPMI-1640 medium containing 10% FBS was added to each well, the plate was placed in the incubator (at 37° C. and 5% CO₂), and incubated for 48 hours.

(2) Purification of HIV-1 and Intracellular Proteins

After 48 hours from the transfection, 500 μL of each culture supernatant was passed through a 0.45 μm filter, and each filtrate containing purified HIV-1 was transferred to a 1.5 mL tube.

With respect to the cells, each well after removing the supernatant was washed with 1 mL of PBS(−) twice, and 0.5 mL of a 0.05% trypsin-EDTA solution was added to detach the cells from the surface of each well. To each well, 0.5 mL of PBS(−) was added, and the whole was mixed. Each cell suspension was transferred to a 1.5 mL tube, and centrifuged at 2000 rpm for 5 minutes at room temperature to remove the supernatant PBS(−). To the precipitated cells in each tube, 300 μL of a solution for cell lysis (PicaGene; Wako Pure Chemical Industries, Ltd., Osaka, Japan) was added to carry out cell lysis. After incubation at 37° C. for 15 minutes, a centrifugation at 13000 rpm was carried out at room temperature for 10 minutes. Each obtained supernatant was transferred to a 1.5 mL tube, and used as a purified solution of intracellular proteins in the following experiments.

(3) Measurement of Amount of p24 Antigen

An amount of an HIV-1 p24 antigen contained in each of the obtained culture supernatants and intracellular proteins was measured by CLEIA (Chemiluminescent Enzyme Immunoassay).

More particularly, each sample was diluted with a commercially available solution for dilution (LUMIPULSE; Fujirebio Inc., Tokyo, Japan) to appropriate concentrations (1/10 to 1/10,000). To 250 μL of each diluted sample solution, 50 μL of a commercially available solution for treating an HIV-1 p24 sample (LUMIPULSE I; Fujirebio Inc., Tokyo, Japan) was added, and the whole was mixed gently. A commercially available reagent (LUMIPULSE f; Fujirebio Inc., Tokyo, Japan) was used to measure an amount of the HIV-1 p24 antigen contained therein.

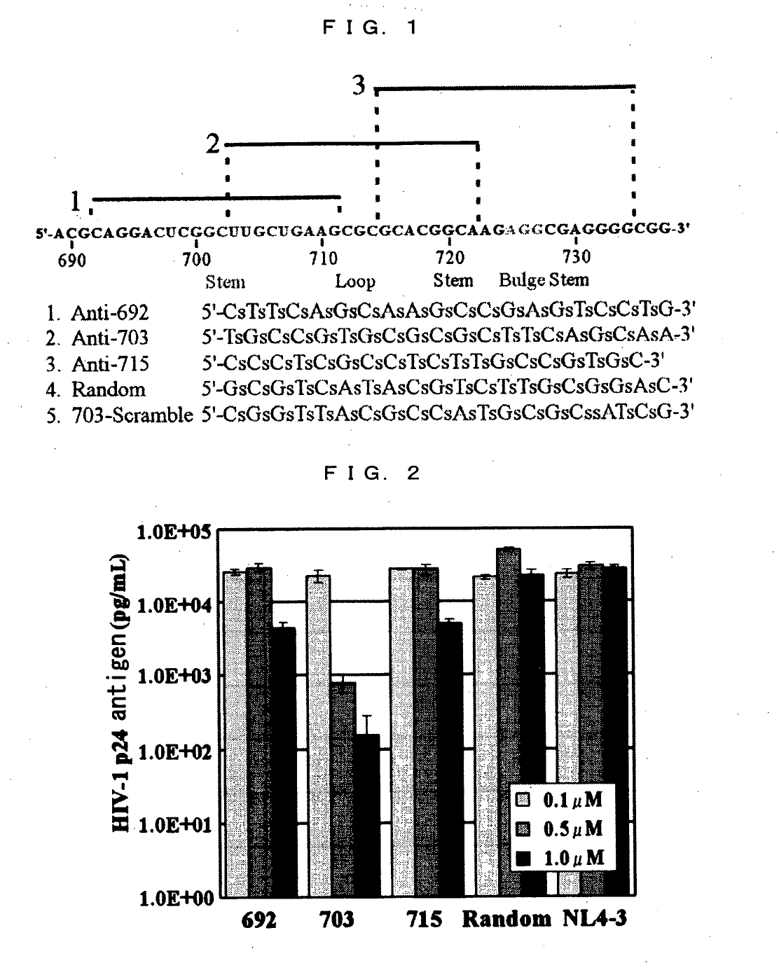

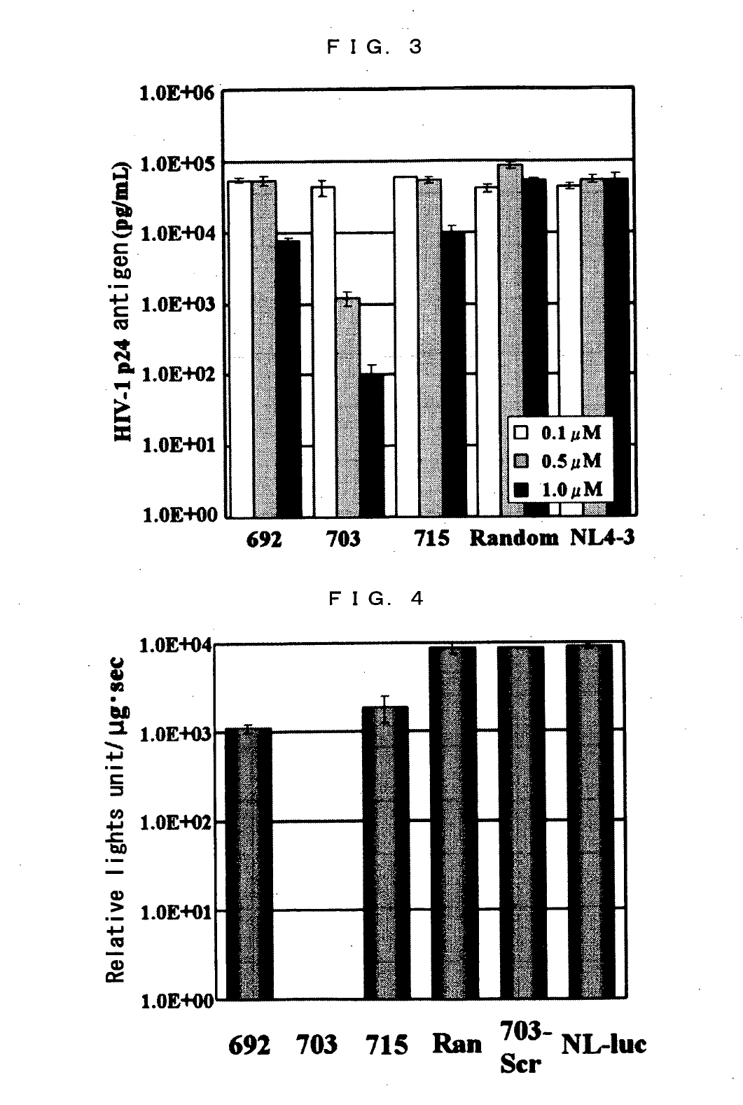

The amounts of the HIV-1 p24 antigen contained in the culture supernatants are shown in FIG. 2, and the amounts of the HIV-1 p24 antigen contained in the intracellular proteins are shown in FIG. 3.

In FIGS. 2 and 3, the “NL4-3” means a control in which no S-ODNs were added.

As shown in FIG. 2, the S-ODN “Anti-703” significantly inhibited the HIV-1 production, and an extremely high activity (99% or more) of inhibiting the HIV-1 production was confirmed. Generally, an antisense nucleic acid needs nucleotides of 17 mer or more as a target sequence, and it is theoretically considered that there is no homological sequence other than the target sequence in the range of 17 mer or more. However, such an antisense nucleic acid may be actually bind to a nucleotide sequence other than the target gene, in accordance with a homology of several nucleotides, and thus problems such as a high cytotoxicity or an unexpected specificity may be caused by suppressing an expression of genes other than the target. In this experiment, the amount of virus production was inhibited by the S-ODN Anti-703, in accordance with the amounts of Anti-703 used for transfection (i.e., 0.1 μmol/L, 0.5 μmol/L, and 1.0 μmol/L), and the result suggested that the Anti-703 inhibited the HIV-1 production by specifically binding to the target sequence.

In addition, the decrease in the amount of p24 contained in the supernatant correlated to the decrease in the amount of p24 contained in intracellular proteins shown in FIG. 3 and the results shows that the inhibitobry activity of Anti-703 for the HIV-1 production inhibits the translation of proteins and the HIV-1 production by specifically recognizing the DIS sequence of HIV-1.

### Example 3

### Evaluation of S-ODNs for Anti-HIV Activity Using Plasmid pNL-luc

(1) Transfection with S-ODNs and Plasmid pNL4-3

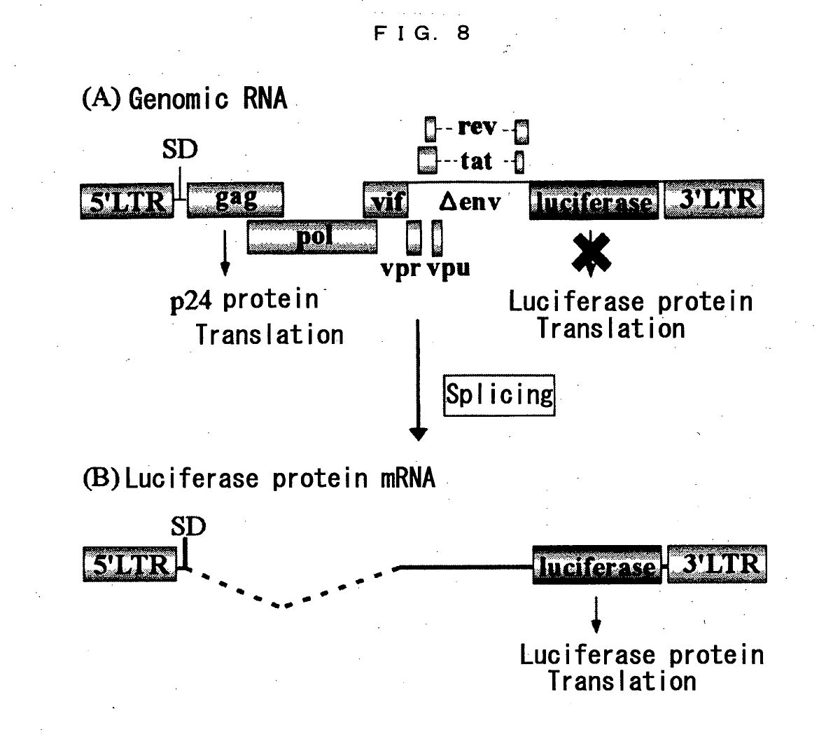

In Example 2, the inhibitory effect of the Anti-703 for the HIV-1 production was shown. Further, it was shown that the inhibitory effect inhibited the production of an HIV-1 protein, p24, by the high antisense activity. As shown in FIG. 8, the HIV-1 p24 protein is a structural protein generated from a Gag precursor protein, and the mRNA thereof is translated from the same RNA as the HIV-1 genomic RNA. Since the target DIS region of HIV-1 is located upstream of SD (see FIG. 7) and exists in all mRNAs of HIV-1, a plasmid pNL-luc was used to examine whether or not the selected S-ODN acts on other mRNAs efficiently.

The plasmid pNL-luc is a plasmid in which a nef region and an env region were deleted and a luciferase gene was inserted instead thereof (see FIG. 8). The HIV-1 genomic RNA transcripted in the nucleus was spliced by a splicing mechanism in the nucleus, to become mRNAs capable of expressing proteins. The p24 antigen evaluated in Example 2 is a gene product translated from the HIV-1 genomic RNA, and the luciferase protein is translated from an mRNA generated by the splicing in the nucleus. The plasmid pNL-luc was used to evaluate the advantage of HIV-1 DIS as the target.

More particularly, the 293T cells (5×10⁵ cells/2 mL medium/well) were added to each well of a 6-well plate, and incubated in an incubator (at 37° C. and 5% CO₂). After 14 hours from the addition, it was confirmed that the cells adhered to the wells. The cell density was approximately 30%.

After the medium was removed from each well, 900 μL of a serum-free D-MEM was added, and the plate was placed in the incubator (at 37° C. and 5% CO₂). After 15 minutes, 100 μL of each FuGENE™6/S-ODN solution, which had been previously prepared by diluting the transfection reagent (FuGENE™6) and each S-ODN with the serum-free D-MEM to predetermined concentrations [S-ODN=10 μmol/L; FuGENE™6=1/50 dilution], was added to each well. The plate was gently shaken to a mix in each well, and placed in the incubator (at 37° C. and 5% CO₂) for two hours.

After two hours had passed from the addition of the FuGENE™6/S-ODN solution, 100 μL of FuGENE™6/pNL-luc solution, which had been previously prepared by diluting the transfection reagent (FuGENE™6) and plasmid pNL-luc with the serum-free D-MEM to predetermined concentrations [plasmid pNL-luc=0.01 μg/μL; FuGENE™6=1/50 dilution], was further added to each well. The plate was gently shaken to a mix in each well, and placed in the incubator (at 37° C. and 5% CO₂) for two hours.

After two hours had passed from the addition of the FuGENE™6/pNL-luc solution, the medium was removed from each well, and 2 mL of D-MEM containing 10% FBS was added to each well. The plate was placed in the incubator (at 37° C. and 5% CO₂), and incubated for 48 hours.

(2) Purification of Intracellular Proteins

After 48 hours from the transfection, the culture supernatant was removed from each well. Each well was washed with 1 mL of PBS(−) twice, 0.5 mL of a 0.05% trypsin-EDTA solution was added thereto, and the plate was incubated in the incubator (at 37° C. and 5% CO₂) for 5 minutes to detach the cells from the surface of each well. To each well, 0.5 mL of PBS(−) was added, and the whole was mixed. Each cell suspension was transferred to a 1.5 mL tube, and centrifuged at 2000 rpm for 3 minutes at room temperature to remove the supernatant PBS(−). In this connection, 50 μL of the cell suspension before the centrifugation was used for counting the cell numbers. The precipitated cells in each tube were suspended in 300 μL of PBS(−), and 150 μL of the cell suspension was transferred to another 1.5 mL tube for use in the following Example 3(4). The remaining cell suspension (150 μL) was centrifuged at 2000 rpm for 5 minutes at room temperature to remove the supernatant PBS(−). To the precipitated cells in each tube, 100 μL of a solution for cell lysis (PicaGene; Wako Pure Chemical Industries, Ltd., Osaka, Japan) was added to carry out cell lysis. A centrifugation at 13000 rpm was carried out at room temperature for 10 minutes. Each obtained supernatant was transferred to another 1.5 mL tube, and used as a purified solution of intracellular proteins.

(3) Measurement of Luciferase Activity in Intracellular Proteins

In this example, 100 μL of a substrate solution for luminescence (Toyo Ink) was added to 10 μL of each purified solution of intracellular proteins, and an luciferase activity in the purified solution of intracellular proteins was measured by a luminometer (LUMAT LB 9507; BERTHOLD).

The result is shown in FIG. 4. In FIG. 4, “Ran”, “703-Scr”, and “NL-luc” mean the S-ODN “Random”, the S-ODN “703-Scramble”, and a control in which no S-ODNs were added, respectively.

As shown in FIG. 4, when an amount of the HIV-1 gene expressed in cells was evaluated as the luciferase activity, the S-ODN which targeted at the DIS of HIV-1 exhibited the inhibitory effect. Particularly, the Anti-703 significantly inhibited the protein expression in this luciferase activity experiment, as well as the inhibition of the HIV-1 p24 antigen expression. It was known that, as a function of antisense nucleic acids, a binding thereof with the target RN hibited progress of-ribosomes in translation and suppressed protein synthesis. It is considered that the efficient inhibitory effect of the S-ODN Anti-703 for the HIV-1 production is caused by an effective actions thereof on the DIS of HIV-1 as an antisense nucleic acid. In this connection, the result suggested that a selection of the site to be targeted by an antisense nucleic acid in a target RNA is important, because other S-ODNs which targeted the DIS of HIV-1 RNA did not exhibit such an efficient inhibitory effect for HIV-1.

In addition, it is known that antisense nucleic acids exhibit a cytotoxicity caused by an interaction thereof with a nonspecific RNA, on the basis of a GC content in the nucleotide sequence. In this example, the 703-Scramble (703-Scr) having the same GC content as that of the Anti-703, which exhibited the inhibitory effect for the HIV-1 production, was evaluated, but did not exhibit the gene expression.

From these results, it was found that the decrease in the amount of HIV-1 by the Anti-703 contributed to the decrease in the amount of the HIV-1 protein synthesis; that the specific recognition to the DIS sequence in the HIV-1 RNA effectively acted on not only the HIV-1 genomic RNA but also the mRNAs, and efficiently inhibited the HIV-1 production and gene expression; and that the DIS region of HIV-1 was a target site useful for the HIV-1 suppression.

(4) Analysis of Amount of Intracellular HIV-1 RNA Expressed

As described above, it was shown that the Anti-703 effectively inhibited the expression of HIV-1 proteins. It is known that, as a function of antisense nucleic acids, an S-ODN or a wild-type antisense nucleic acid and the target RNA form a duplex, which is a substrate of intracellular RNase H, and that the target RNA is digested. In this example, an amount of intracellular HIV-1 RNA was analyzed to examine an RNase H activity to the target RNA with respect to the selected S-ODNs.

More particularly, a commercially available RNA extraction reagent (TRIZOL™ Reagent; Invitrogen Japan K.K.) was used to extract intracellular RNA from the cell suspension obtained in Example 3(2). The total RNA was treated with DNase I, and a reverse transcriptase-polymerase chain reaction (RT-PCR) was carried out. As the primers for RT-PCR, primers capable of specifically amplifying the gag region of HIV-1 RNA were used. As primers for an internal control, primers capable of amplifying a glyceraldehyde-3-phosphate dehydrogenase (G3PDH) gene were used.

The result is shown in FIG. 5. In FIG. 5, “M” means a DNA marker; “NL-luc” means a control in which the plasmid pNL-luc was added, but no S-ODNs were added; “N.C.” means a control in which the plasmid pNL-luc was not added, and no S-ODNs were added; and “RT(−)pNL-luc” means a control in which the plasmid pNL-luc was added, but the reverse transcriptase was not added.

As shown in FIG. 5, when any one of the S-ODN “Random”, the S-ODN “703-Scramble”, or the plasmid pNL-luc was transfected singly, no change in the fluorescence of each band was observed. In contrast, a decreased amount of the HIV-1 RNA gene expressed was observed in the S-ODN which targeted the DIS of HIV-1, that is, the RNA gene was not amplified by the RT-PCR in the Anti-703.

From the result, it is considered that the effective inhibitory effect of the Anti-703 for the HIV-1 production is caused by the inhibition of the intracellular protein synthesis and RNA digestion by binding to the target RNA, i.e., by the function of the Anti-703 as antisense nucleic acids.

As described above, it was found that the inhibitory effect of the selected S-ODN for the HIV-1 production was caused by the decrease of the HIV-1 protein expression and the decrease of the amount of RNA, and that the S-ODN (Anti-703) specifically bound to the target RNA (DIS in HIV-1) and inhibited the production of viral particles. Further, from the analysis of HIV-1 protein expression, it was confirmed that the DIS region of HIV-1 was important for not only genomic RNA but also mRNAs, and was a effective target site in the antisense method.

### Example 4

### Confirmation of Anti-HIV-1 Activity Using Human Peripheral Blood Lymphocytes (1)

In this example, the Anti-703 used in Examples 2 and 3, an Anti-694 (SEQ ID NO: 3), which targeted at a sequence in which the target sequence of the Anti-703 was shifted by 10 nucleotides to the 5′ direction, and an Anti-713 (SEQ ID NO: 5), which targeted at a sequence in which the target sequence of the Anti-703 was shifted by 10 nucleotides to the 3′ direction, were evaluated using human peripheral blood lymphocytes, with respect to the anti-HIV-1 activity thereof.

Human lymphocytes were separated from peripheral blood taken from a healthy person. An HIV-1 NL432 stain (Adachi, A. et al., J. Virol., 59, 284, 1986) was added to the lymphocytes, and the mixture was allowed to stand on ice for 90 minutes. The lymphocytes were washed with an RPMI-1640 medium to remove any non-adsorbed virus. Each S-ODN (0.5 μmol/L) such as Anti-703 was mixed with a transfection reagent (DMRIE-C; Invitrogen), and each mixture was added to the infected lymphocytes. The cells were cultivated while the medium was changed every 4 days After 14 days, an amount of the viral protein HIV-1 p25 antigen produced in the culture supernatant was measured.

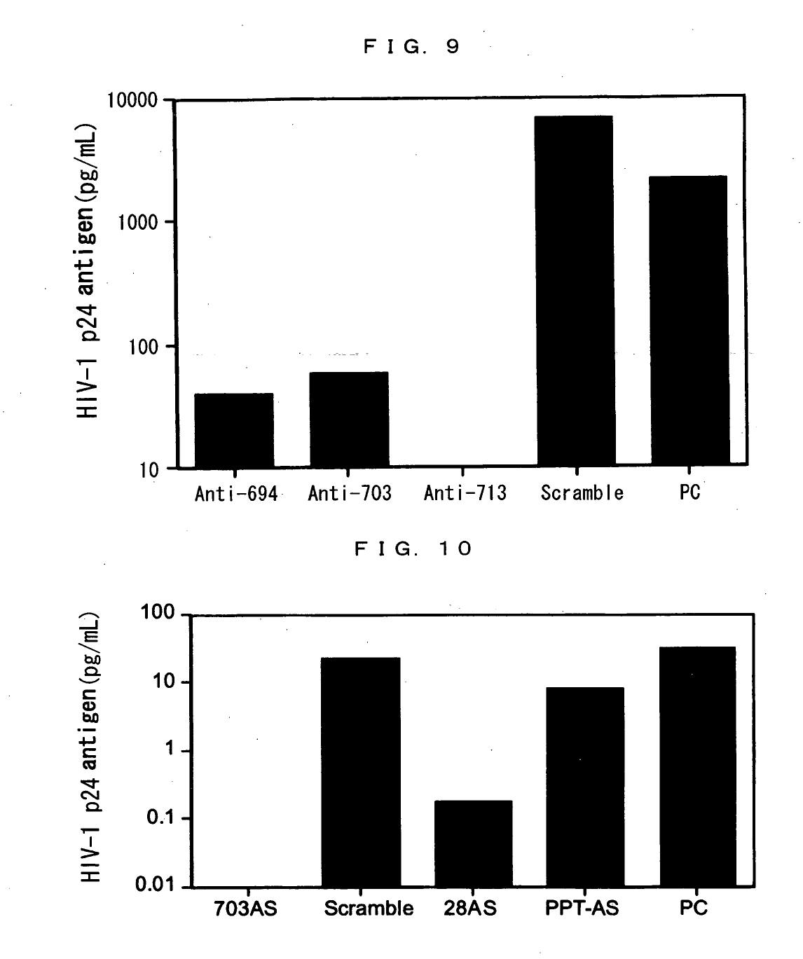

The result is shown in FIG. 9. In FIG. 9, “PC” means a positive control in which no S-ODNs were added.

As shown in FIG. 9, three S-ODNs other than the S-ODN (Scramble) having a scramble sequence of the Anti-703, i.e., Anti-703, Anti-694, and Anti-713, exhibited an extremely high antiviral activity in comparison with the positive control. The result shows that these S-ODNs can inhibit the viral infection to human peripheral blood lymphocytes.

### Example 5

### Confirmation of Anti-HIV-1 Activity Using Human Peripheral Blood Lymphocytes (2)

The procedure described in Example 4 was repeated, except that an S-ODN 28AS (SEQ ID NO: 9) which targeted the gag region, and an S-ODN PPT-AS (SEQ ID NO: 10) which targeted a polypurine tract region important for a reverse transcription of viral genes were used instead of the Anti-694 and the Anti-713.

The result is shown in FIG. 10. In FIG. 10, “703AS” means the Anti-703, and “PC” means a positive control in which no S-ODNs were added.

The 28AS which targeted the gag region, or the PPT-AS which targeted the polypurine tract region important for a reverse transcription of viral genes, did not inhibit the viral protein expression, in comparison with the Anti-703 which targeted the DIS region.

### INDUSTRIAL APPLICABILITY

The oligonucleotide of the present invention is useful as an active ingredient of an anti-HIV agent. According to the anti-HIV agent of the present invention, an HIV infection may be effectively treated and/or prevented.

Free Text in Sequence Listing

In each of nucleotide sequences of SEQ ID NOS: 2 to 10, all internucleotide bonds are phosphorothioate bonds.

Each of the nucleotide sequences of SEQ ID NOS: 7 and 8 is an artificially synthesized random sequence.

Although the present invention has been described with reference to specific embodiments, various changes and modifications obvious to those skilled in the art are possible without departing from the scope of the appended claims.

1. An oligonucleotide consisting of a nucleotide sequence complementary to a nucleotide sequence consisting of at least 15 successive nucleotides in the nucleotide sequence consisting of nucleotides 6-44 of SEQ ID NO: 1.

2. An oligonucleotide comprising a nucleotide sequence which specifically hybridizes to a nucleotide sequence consisting of at least 15 successive nucleotides in the nucleotide sequence consisting of nucleotides 6-44 of SEQ ID NO: 1.

3. The oligonucleotide according to claim 1, wherein at least one internucleotide bond is a phosphorothioate bond.

4. A pharmaceutical composition comprising the oligonucleotide according to claim 1, and a pharmaceutically acceptable carrier or diluent.

5. An anti-HIV agent comprising the oligonucleotide according to claim 1.

6. A pharmaceutical composition for treating or preventing HIV, comprising the oligonucleotide according to claim 1, and a pharmaceutically or veterinarily acceptable carrier or diluent.

7. A method for treating or preventing HIV, comprising administering to a subject in need thereof the oligonucleotide according to claim 1 in an amount effective therefor.

8. (canceled)

9. The oligonucleotide according to claim 2, wherein at least one internucleotide bond is a phosphorothioate bond.

10. A pharmaceutical composition comprising the oligonucleotide according to claim 2, and a pharmaceutically acceptable carrier or diluent.

11. A pharmaceutical composition comprising the oligonucleotide according to claim 3, and a pharmaceutically acceptable carrier or diluent.

12. An anti-HIV agent comprising the oligonucleotide according to claim 2.

13. An anti-HIV agent comprising the oligonucleotide according to claim 3.

14. A pharmaceutical composition for treating or preventing HIV, comprising the oligonucleotide according to claim 2, and a pharmaceutically acceptable carrier or diluent.

15. A pharmaceutical composition for treating or preventing HIV, comprising the oligonucleotide according to claim 3, and a pharmaceutically acceptable carrier or diluent.

16. A method for treating or preventing HIV, comprising administering to a subject in need thereof the oligonucleotide according to claim 2 in an amount effective therefor.

17. A method for treating or preventing HIV, comprising administering to a subject in need thereof the oligonucleotide according to claim 3 in an amount effective therefor.
